(19) United States
(12) Patent Application Publication
(10) Pub. No.: US 2022/0323454 A1
(43) Pub. Date: Oct. 13, 2022

(54) PHENOTHIAZINES AND THEIR DERIVATIVES FOR USE AS A MEDICAMENT

(71) Applicant: Guangzhou Virotech Pharmaceutical Co., Ltd., Guangzhou (CN)

(72) Inventors: Liang Chen, Guangzhou (CN); Zhenzhen Fan, Guangzhou (CN); Songmin He, Guangzhou (CN); Jiesi Chen, Guangzhou (CN); Max Sander, Guangzhou (CN); Shoufang Gong, Guangzhou (CN); Xiaozhi Yang, Guangzhou (CN); Ziqing Lin, Guangzhou (CN)

(21) Appl. No.: 17/638,663

(22) PCT Filed: Aug. 25, 2020
(86) PCT No.: PCT/CN2020/110998

§ 371 Date: Feb. 25, 2022

(30) Foreign Application Priority Data
Aug. 26, 2019 (CN) 201910792478.5

(51) Int. Cl. A61K 31/5415; A61K 35/17; A61K 39/395; A61P 35/00

(52) U.S. Cl. CPC A61K 31/5415; A61K 35/17; A61K 39/3955; A61P 35/00

(57) ABSTRACT

Disclosed is a new use of a phenothiazine compound in pharmacy, particularly in the preparation of a PD-1 signaling inhibitor, which is selected from the compounds represented by formula (I), their hydrates, solvates and reduced forms. The phenothiazine compounds disclosed herein inhibit the function of PD-1 and block the signal transduction of PD-1, so that they can be used as PD-1 signal transduction inhibitors. In vitro experiments found that these compounds can restore the function of immune cells inhibited by PD-1, thereby improving the function of CTL and other immune cells to secrete cytokines and kill target cells, enhancing the body's immune function and being effective in treating tumor.

### TECHNICAL FIELD

The present invention is in the field of medical technology, and relates, in particular, to phenothiazines, their derivatives, hydrates, solvates or reduced forms for use as a medicament. The present invention further relates to the combinational use of phenothiazines with adoptive cell therapy. The present invention further relates to the combinational use of phenothiazines with tumor immunotherapy such as an anti-PD-1 antibody.

### BACKGROUND

Adoptive cell therapy is a therapy including collecting one or more different types of immune cells from a mammal, ex vivo culturing and/or manipulating the collected immune cells, and returning the cultured and/or manipulated immune cells back to the mammal Ex vivo manipulation of the collected immune cells includes introduction of a recombinant nucleic acid into the immune cells. Adoptive cell therapy includes but is not limited to tumor infiltrating lymphocytes (TIL), lymphokine activated killer (LAK) cells, cytokine induced killer (CIK) cells, dendritic cells (DCs), natural killer (NK) cells, T cell receptor-modified T cell (TCR-T), CAR-NK, chimeric antigen receptor engineered T (CAR-T) cells, and the like.

Human immune system can defend and remove foreign invading pathogenic microorganisms or mutated cells, and immune cells are important cells that perform immune function. Among them, T cells are important immune cells in human body. They can recognize antigens presented by the major histocompatibility complex (MHC) on target cells through T cell receptors (TCR) expressed on the surfaces, which is known as the first signal. T cells are activated and function by co-stimulatory secondary signal (such as B7 molecule) on the target cell. For example, one of the subtypes of T cells, cytotoxic T cells (CTL), can directly kill target cells after being activated, thereby eliminating virus-infected cells or tumor cells.

In the process of T cell activation, T cells initiate the expression of some molecules that inhibit their immune function by negative feedback, and PD-1 is one of the most famous molecules. When stimulated by its ligand, usually PD-L1 on target cells, such as tumor cells, PD-1 protein on the surface of CTL cells initiates signal transduction, inhibits the function of CTL, and results in T cell apoptosis or anergy. Such signaling makes CTLs lose their ability to kill target cells. Tumor cells usually express PD-L1 on the cell surface, inhibits the killing of CTL and thereby escapes immune surveillance. When PD-1 signaling was blocked, the ability of CTL to kill tumor cells was rescued. Immune cells other than T cells can also express PD-1, such as macrophages and B cells. When the activity of PD-1 is inhibited, the function of the immune cells is partly restored. For example, when PD-1 pathway is blocked, macrophages switch from M2 (the type that inhibits CTL function) to M1 (the type that promotes CTL function) and eliminate tumor cells from the body. Biological agents such as anti-PD-1 antibodies have been approved by the FDA for clinical treatment. However, up to now, the drugs targeting PD-1 in clinical use are all antibodies of biological agents, while PD-1 signal transduction inhibitors of small molecule compounds have not been reported.

Methylene blue (3,7-bis(dimethylamino)-phenothiazine-5-ium chloride) is a phenothiazine salt widely used as chemical indicators, dyes, and biological stains. Recent studies showed its application in medicine. For example, CN 104027338 A disclosed the use of methylene blue in treating acute cerebral ischemia and CN 103417546 B disclosed its use in postanesthetic recovery. CN 106668859 A disclosed that methylene blue was used as a photosensitizer for photodynamic therapy. Traditional photodynamic therapy was found attenuation of excitation light intensity for in vivo treatment due to the absorption and scattering of the light by biological tissues. The hypoxic status of malignant tumor tissues leads to low yield of singlet oxygen, and a pure photosensitizer does not actually treat tumor.

There is no report regarding use of methylene blue in the inhibition of PD-1 signaling and prevention or treatment of a tumor, despite the disclosure of its uses in medicine. Drugs for cancer treatment are still clinically demanded, so it is of great clinical significance to provide a drug that can effectively treat cancer.

### SUMMARY

The present invention provides a pharmaceutical composition, comprising (a) an immune cell for adoptive cell therapy, and (b) a phenothiazine compound having formula (I), or a hydrate, a solvate or a reduced form thereof,

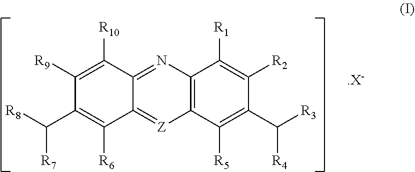

wherein,

Z is selected from a group consisting of S⁺, O⁺, C and N;

Y is N or N⁺; when Z is S⁺ or O⁺, Y is N; and when Z is C or N, Y is N⁺;

X⁻ is one or more anions that form a salt with Z⁺ or N⁺ to achieve electric neutrality; and

R₁, R₂, R₃, R₄, R₅, R₆, R₇, R₈, R₉ and R₁₀ are independently selected from a group consisting of hydrogen, substituted or unsubstituted alkyl, substituted or unsubstituted cycloalkyl, substituted or unsubstituted heterocyclic alkyl, substituted or unsubstituted aryl, substituted or unsubstituted heteroaryl, substituted or unsubstituted alkoxyl, substituted or unsubstituted arylalkoxyl, thioalkoxyl, amido, nitro, amino, and halogen.

In some embodiments, X is an inorganic anion or an organic anion. The inorganic anion is preferably Cl⁻, Br⁻ or I⁻. The organic anion is preferably methanesulfonic ion, ethanesulfonic ion, p-toluenesulfonic ion, benzenesulfonic ion, ethanedisulfonic ion, propanedisulfonic ion, or naphthalene disulfonic ion.

In some embodiments, R₁, R₂, R₃, R₄, R₅, R₆, R₇, R₈, R₉ and R₁₀ are independently selected from a group consisting of hydrogen, substituted or unsubstituted C1-C₆ alkyl, substituted or unsubstituted C₃-C₅ cycloalkyl, substituted or unsubstituted 3- to 8-member heterocyclic alkyl, substituted or unsubstituted C₅-C₁₀ aryl, substituted or unsubstituted 5- to 10-member heteroaryl, substituted or unsubstituted C1-C₆ alkoxyl, substituted or unsubstituted arylalkoxyl, thioalkoxyl, amido, nitro, amino, and halogen. In some embodiments, the phenothiazine compound is 3,7-bis(dimethylamino)-phenothiazine-5-ium chloride.

In some embodiments, the phenothiazine compound is 3,7-bis(dimethylamino)-phenothiazine-5-ium salt (MTX) having formula (II), or a hydrate, a solvate, or a reduced form thereof,

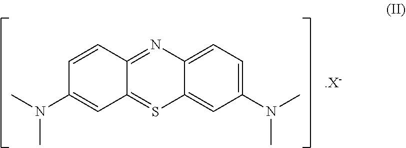

wherein X⁻ is one or more anion as defined above, to achieve electric neutrality.

In some embodiments, the reduced form of the phenothiazine compound is N3,N3,N7,N7-tetramethyl-10H-phenothiazine-3,7-diammonium salt (LMTX) having formula (III), or a hydrate or a solvate thereof,

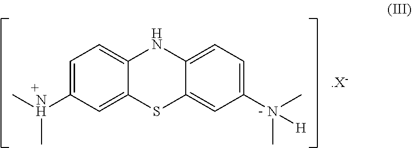

wherein X⁻ is one or more anion as defined above, to achieve electric neutrality.

In some embodiments, the phenothiazine compound is 3,7-bis(dimethylamino)-phenothiazine-5-ium chloride or N3,N3,N7,N7-tetramethyl-10H-phenothiazine-3,7-diammonium dimesylate.

In some embodiments, the immune cell is selected from a group consisting of a tumor infiltrating lymphocyte (TIL), a chimeric antigen receptor T cell (CAR-T), a chimeric antigen receptor NK cell (CAR-NK) and a T cell receptor (TCR) chimeric T cell (TCR-T).

In some embodiments, the immune cell is a T cell receptor (TCR) chimeric T cell (TCR-T).

In some embodiments, the TCR can bind to SIINFEKL peptide.

A further aspect of the invention provides a kit comprising (a) any of the immune cell as described herein, prepared into a first formulation, and (b) any of the phenothiazine compound as described herein, prepared into a second formulation.

A further aspect of the invention provides a use of any of the phenothiazine compound as described herein in the preparation of a medicament for treating cancer.

In some embodiments, the cancer is selected from a group consisting of melanoma, thymic tumor, lung cancer, prostate cancer, breast cancer, ovarian cancer, colorectal cancer, pancreatic cancer, liver cancer, lymphoma, esophageal cancer, bladder cancer, urethral cancer, non-Hodgkin's lymphoma, kidney cancer and brain tumor.

A further aspect of the invention provides a use of any of the phenothiazine compound as described herein in the preparation of a medicament for boosting immune cell function.

A further aspect of the invention provides a use of any of the phenothiazine compound as described herein in the preparation of a medicament for inhibiting PD-1 signaling. A further aspect of the invention provides a use of any of the phenothiazine compound as described herein in the preparation of a medicament for blocking PD-1 downstream signaling pathway. A further aspect of the invention provides a use of any of the phenothiazine compound as described herein in the preparation of a medicament for blocking PD-1 from recruiting SHP2 protein.

A further aspect of the invention provides a method for inhibiting PD-1 signaling comprising a step of blocking PD-1 downstream signaling pathway. In some embodiments, the blocking is achieved through blocking PD-1 from recruiting SHP2 protein. In some embodiments, the blocking PD-1 from recruiting SHP2 protein comprises contacting a cell with an effective amount of any of the phenothiazine compound as described herein.

A further aspect of the invention provides a method for treating a caner in a subject, comprising inhibiting PD-1 signaling in the subject. In some embodiments, the inhibiting PD-1 signaling comprises blocking PD-1 downstream signaling pathway. In some embodiments, the blocking is achieved through blocking PD-1 from recruiting SHP2 protein. In some embodiments, the blocking PD-1 from recruiting SHP2 protein comprises administering to the subject an effective amount of any of the phenothiazine compound as described herein.

A further aspect of the invention provides a method for treating a caner in a subject, comprising administering to the subject an effective amount of any of the phenothiazine compound as described herein and a second therapy. In some embodiments, the second therapy is selected from a group consisting of chemotherapy, radiotherapy and surgery. In some embodiments, the chemotherapy is tumor immunotherapy. In some embodiments, the tumor immunotherapy comprises administering an anti-PD-1 antibody, an anti-PD-L1 antibody, or a functional fragment thereof. In some embodiments, the methods of this aspect provide synergistic anti-tumor effects.

A further aspect of the invention provides a pharmaceutical composition for treatment of cancer, comprising any of the phenothiazine compound as described herein and a second chemotherapeutic agent. In some embodiments, the second chemotherapeutic agent is a tumor immunotherapeutic agent. In some embodiments, the tumor immunotherapeutic agent is an anti-PD-1 antibody, an anti-PD-L1 antibody, or a functional fragment thereof.

A further aspect of the invention provides a method for improving the efficacy of an anti-PD-1 antibody, an anti-PD-L1 antibody, or a functional fragment thereof in a subject, comprising administering to the subject an effective amount of the phenothiazine compound as described herein before, simultaneously, or after the administration of the anti-PD-1 antibody, the anti-PD-L1 antibody, or the functional fragment thereof. In some embodiments, the methods of this aspect provide synergistic anti-tumor effects.

The present inventors creatively found that the phenothiazines and derivatives disclosed herein can significantly and synergistically improve the killing of target cells by immune cells when used in combination with the immune cells, especially genetically modified immune cells. The phenothiazines and their derivatives disclosed herein inhibit the function of PD-1 and block the signal transduction of PD-1, thereby they can be used as PD-1 signal transduction inhibitors.

In vitro experiments found that the compounds disclosed herein can restore the function of immune cells inhibited by PD-1, thereby improving the function of immune cells such as CTL to secrete cytokines and kill target cells, thereby improving the immune function of the body. Animal models showed that the phenothiazine compounds reduced transplanted tumors or in situ lung cancers in mice. In addition, compared with biomolecules such as PD-1 antibodies, small molecule PD-1 signaling inhibitors have the advantages of lower cost, simpler preparation process (such as through chemical synthesis), multiple routes of administration, high patient compliance, safety and reliability, and etc. The phenothiazine compounds disclosed herein were shown to have an equivalent or even better tumor inhibition effect than PD-1 antibodies.

### BRIEF DESCRIPTION OF THE DRAWINGS

FIG. 1 shows the results of the experiments of example 1, in which A shows the expression of PD-1 protein on untransfected and transfected Jurkat cell surfaces, B shows the expression of PD-L1 protein on the untransfected and transfected Raji cell surfaces, C shows the Y248 phosphorylation of PD-1 in transfected Jurkat cells induced by transfected Raji cells, D shows the levels of IL-12 secreted by Jurkat-PD-1-NFAT-luc cells stimulated by MTC, and E shows the increase of luciferase activity in Jurkat-PD-1-NFAT-luc cells in the presence of MTC or LMT (N3,N3,N7,N7-tetramethyl-10H-phenothiazine-3,7-diammonium dimesylate).

FIG. 2 shows OT-1 cell division stimulated by MTC, in which A shows the expression of PD-1 on OT1-CTL cells, and B shows MTC stimulated CTL cell division.

FIG. 3 shows MTC stimulated effector molecules secretion by OT-1 cells, in which the secretion of IL-2, IFNγ, Perforin and Granzyme B were enhanced.

FIG. 4 shows the capacity of target cell killing of OT-1 T cells was restored by MTC, in which A shows MTC promoted killing of EL4-OVA (PD-L1) by CTL, B shows IFNγ-induced PD-L1 expression by B16-F10, C and D show MTC or LMT synergistically enhanced killing of B16-OVA cells by CTL, and E shows MTC or LMT had no killing effect on B16-WT cells, even in combination with CTL cells.

FIG. 5 shows the results of MTC promoted clearance of tumors formed by target cells by cytotoxic T cells (CTL), in which A is the experiment protocol, B to D show MTC inhibited xenograft growth in Rag1−/− mice, and E shows MTC inhibited xenograft growth in C57BL/6J mice.

FIG. 6 shows the results of MTC treatment of primary tumors, in which A is the experiment protocol, and B shows MTC stimulated clearance of tumor through CD8⁺ T cells.

FIG. 7 shows MTC blocked PD-1 from recruiting SHP2, in which A shows MTC reduced binding of PD-1 to SHP2, B shows MTC inhibited PD-1 from recruiting SHP2, and C shows MTC inhibited the binding of PD-1 with SHP2.

### DETAILED DESCRIPTION OF THE INVENTION

The term “adoptive cell therapy” or ACT as used herein involves transfer of anti-tumor immune cells to a cancer patient. In some examples, ACT is a therapy involving isolating tumor-specific lymphocytes, in vitro expanding the cells to large amount and infusing the cells to a cancer-bearing host.

The term “tumor infiltrating lymphocyte” or TIL as used herein refers to a white blood cell that has left the bloodstream and migrated into a tumor.

The term “CAR-T” is an abbreviation for chimeric antigen receptor T cell, in which the chimeric antigen receptor (CAR) is the core moiety of the CAR-T, conferring T cells with an ability to recognize an antigen of a target cell, such as a tumor cell, in an HLA independent manner, so that the CAR engineered T cells recognize a broader range of targets than native TCRs can.

The term “TCR-T” or “T cell receptor chimeric T cell” as used herein refers to a T cell expressing an engineered or artificial T cell receptor (TCR). The engineered or artificial TCR is genetically engineered to target an antigen of interest while domains and/or accessory molecules for TCR signaling are retained. In certain examples, TCR-T retains all accessory molecules for TCR signaling, so that it is fully activated at very low level of antigen stimulation and induces killing effects against target cells. TCR-T has higher sensitivity in recognizing antigens present at low concentration and low copy numbers than CAR-T, suggesting great therapeutic potential.

Protein tyrosine phosphatase non-receptor type 11 (PTPN11), also known as protein tyrosine phosphatase 1D (PTP-1D), Src homology-2 domain-containing protein tyrosine phosphatase (SHP2), or protein tyrosine phosphatase 2C (PTP-2C), is an enzyme encoded by ptpn11 gene. SHP-2 is ubiquitously expressed in various tissues and cell types and is involved in multiple signaling pathways, including growth factors such as PDGF, EGF, and IGF-1, cytokines such as IL-3, GM-CSF, and EPO, insulin, and interferons. SHP-2 has complicated signaling functions, which appears to be involved in multiple signal transduction processes, such as the Ras-Raf-MAP-ERK pathway, the Jak-Stat pathway and the PI3K-Akt pathway. It has also been shown to bind to various signaling intermediates such as Grb2, FRS2, Jak2, the p85 subunit of PI3 kinase, IRS-1, Gab1 and Gab2. As a downstream molecule of the PD-1 receptor, SHP-2 is involved in the transduction of T cell inhibitory signaling. Studies have shown that SHP-2 is a downstream molecule of PD-1 signaling, which not only inhibits T cell activation but also promotes T cell anergy. Previous studies have also shown that knockout of SHP2 in T lymphocytes can trigger anti-tumor immunity and inhibit the occurrence of colitis-related cancers in mice.

Methylthionine (MT) is a redox molecule and exists in a state of equilibrium between the reduced form of 10H-phenothiazine (i.e., N3,N3,N7,N7-tetramethyl-10H-phenothiazine-3,7-diammonium, LMT) and the oxidized form (MT+).

The salt of oxidized form (MTX) has formula (II),

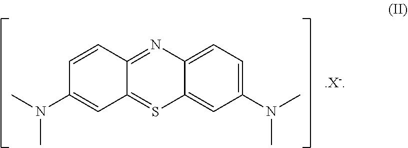

When LMT exists in its salt form it is known as LMTX salt, which has formula (III),

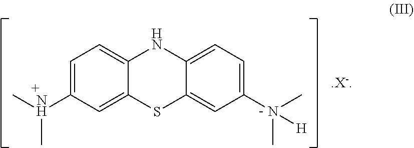

X⁻ is an inorganic anion or an organic anion. Suitable organic anion examples include but are not limited to those organic anions derived from organic acids selected from a group consisting of 2-acetoxybenzoic acid, acetic acid, ascorbic acid, aspartic acid, benzoic acid, camphorsulfonic acid, cinnamic acid, citric acid, EDTA, ethanedisulfonic acid, ethanesulfonic acid, fumaric acid, glucoheptonic acid, gluconic acid, glutamic acid, glycolic acid, hydroxymaleic acid, hydroxynaphthoic acid, isethionic acid, lactic acid, lactobionic acid, lauric acid, maleic acid, malic acid, methanesulfonic acid, mucic acid, oleic acid, oxalic acid, palmitic acid, pamoic acid, pantothenic acid, phenylacetic acid, benzenesulfonic acid, propanedisulfonic acid, propionic acid, pyruvic acid, salicylic acid, stearic acid, succinic acid, p-aminobenzenesulfonic acid, tartaric acid, toluenesulfonic acid, p-toluenesulfonic acid, naphthalenedisulfonic acid and valeric acid. Suitable polymeric organic anions include but are not limited to those polymeric organic anions derived from polymeric acids selected from a group consisting of tannic acid and carboxymethyl cellulose. The inorganic anion is preferably Cl⁻, Br⁻ or I⁻. The organic anion is preferably methanesulfonic anion.

Methylene blue (MTC), also referred to herein as 3,7-bis(dimethylamino)-phenothiazine-5-ium chloride, is a chloride of the oxidized form (i.e., MT+) of MT, which is a soluble tricycle compound with low molecule weight (319.86) and formula of,

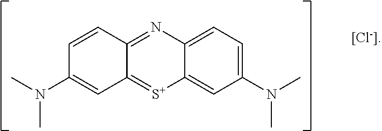

N3,N3,N7,N7-tetramethyl-10H-phenothiazine-3,7-diammonium dimesylate has formula of,

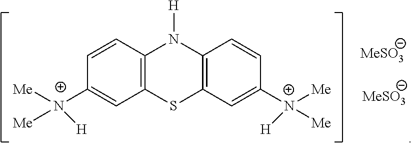

The term “solvate” as used herein has the meaning as normally understood in the art, which is a complex of a solute (such as a compound or a salt of a compound) and a solvent. If the solvent is water, the solvate can conveniently be referred to as hydrate, such as monohydrate, dihydrate, trihydrate and the like. Unless specified otherwise, when a specific compound is mentioned, the specific compound also comprises its solvate.

The term “treatment” as used herein includes administration of a compound or a composition of the present invention to alleviate symptoms or complications of a disease or disorder, or to eliminate a disease or disorder. As used herein, the term “alleviate” is used to describe a process of reducing the severity of signs or symptoms of a disorder. Symptoms can be relieved but not eliminated. In one embodiment, administration of a composition of the present invention results in the elimination of signs or symptoms.

Uses and Therapies

An aspect of the present invention is related to use of a phenothiazine compound in the preparation of a PD-1 signal transduction inhibitor, wherein the phenothiazine compound is a compound having formula (I), or a hydrate, a solvate, or a reduced form thereof,

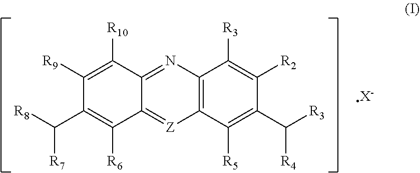

wherein,

Z is selected from a group consisting of S⁺, O⁺, C and N;

Y is N or N⁺; when Z is S⁺ or O⁺, Y is N; and when Z is C or N, Y is N⁺;

X⁻ is one or more anions that form a salt with Z⁺ or N⁺ to achieve electric neutrality; and

R₁, R₂, R₃, R₄, R₅, R₆, R₇, R₈, R₉ and R₁₀ are independently selected from a group consisting of hydrogen, substituted or unsubstituted alkyl, substituted or unsubstituted cycloalkyl, substituted or unsubstituted heterocyclic alkyl, substituted or unsubstituted aryl, substituted or unsubstituted heteroaryl, substituted or unsubstituted alkoxyl, substituted or unsubstituted arylalkoxyl, thioalkoxyl, amido, nitro, amino, and halogen.

In some embodiments, the phenothiazine compound is 3,7-bis(dimethylamino)-phenothiazine-5-ium salt (MTX) having formula (II), or a hydrate, a solvate, or a reduced form thereof,

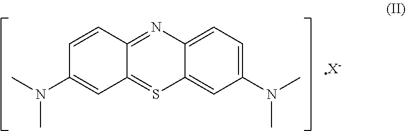

wherein X is one or more anion as defined above, to achieve electric neutrality.

In some embodiments, the reduced form of the phenothiazine compound is N3,N3,N7,N7-tetramethyl-10H-phenothiazine-3,7-diammonium salt (LMTX) having formula (III), or a hydrate or a solvate thereof,

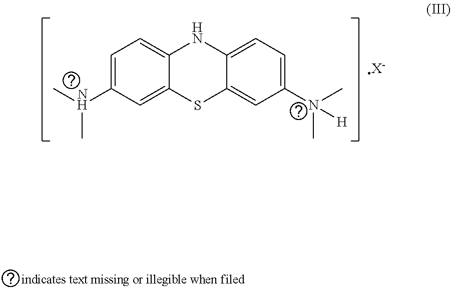

wherein X is one or more anion as defined above, to achieve electric neutrality.

In some embodiments, the phenothiazine compound is 3,7-bis(dimethylamino)-phenothiazine-5-ium salt (MTX), preferably 3,7-bis(dimethylamino)-phenothiazine-5-ium chloride.

Another aspect of the present invention is related to use of the phenothiazine compound of formula I, or a hydrate, a solvate, or a reduced form thereof, in the preparation of a medicament for treatment of cancer. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate.

Another aspect of the present invention is related to use of the phenothiazine compound of formula I, or a hydrate, a solvate, or a reduced form thereof, in the preparation of a medicament for boosting immune cell function. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate.

Another aspect of the present invention is related to use of the phenothiazine compound of formula I, or a hydrate, a solvate, or a reduced form thereof, in the preparation of a medicament for prevention or treatment of cancer recurrence. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate.

Another aspect of the present invention is related to use of the phenothiazine compound of formula I, or a hydrate, a solvate, or a reduced form thereof, in the preparation of a medicament for blocking PD-1 downstream signal pathway. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate.

Another aspect of the present invention is related to use of the phenothiazine compound of formula I, or a hydrate, a solvate, or a reduced form thereof, in the preparation of a medicament for blocking PD-1 from recruiting SHP2 protein. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate.

Another aspect of the present invention is related to a method for treating cancer, comprising administering to a subject a therapeutically effective amount of a phenothiazine compound of formula I, or a hydrate, a solvate, or a reduced form thereof. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate. In some embodiments, the cancer is selected from a group consisting of melanoma, thymic tumor, lung cancer, prostate cancer, breast cancer, ovarian cancer, colorectal cancer, pancreatic cancer, liver cancer, lymphoma, esophageal cancer, bladder cancer, urethral cancer, non-Hodgkin's lymphoma, kidney cancer and brain tumor. In some embodiments, the cancer is melanoma. In some embodiments, the subject is a mammal, preferably a human being.

Another aspect of the present invention is related to a method for boosting immune cell function, comprising administering to a subject a therapeutically effective amount of a phenothiazine compound of formula I, or a hydrate, a solvate, or a reduced form thereof. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate. In some embodiments, the boosting immune cell function achieves antiviral effects. In some embodiments, the subject is a mammal, preferably a human being.

Another aspect of the present invention is related to a method for prevention or treatment of cancer recurrence, comprising administering to a subject a therapeutically effective amount of a phenothiazine compound of formula I, or a hydrate, a solvate, or a reduced form thereof. In some embodiments, the phenothiazine compound is 3,7-bis(dimethylamino)-phenothiazine-5-ium salt (MTX) of formula II, or a hydrate, a solvate, or a reduced form thereof. In some embodiments, the phenothiazine compound is N3,N3,N7,N7-tetramethyl-10H-phenothiazine-3,7-diammonium salt (LMTX). In this aspect, preferably, the phenothiazine compound is 3,7-bis(dimethylamino)-phenothiazine-5-ium chloride or N3,N3,N7,N7-tetramethyl-10H-phenothiazine-3,7-diammonium dimesylate. In some embodiments, the subject has undergone surgery, chemotherapy or radiotherapy.

In some embodiments, the methods can be used in combination with other therapies for cancers. For example, the methods can be used in combination with surgery, radiotherapy or chemotherapy. Accordingly, in some embodiments, the methods of treating cancer of the present invention further comprise administering to the subject suffering from said cancer a therapeutically effective amount of a second therapeutic agent.

In some embodiments, the second therapeutic agent is an immune cell suitable for adoptive cell therapy. In these embodiments, the second therapeutic agent is administered prior to, concurrently with, or subsequent to administration of the phenothiazine compound of formula I, or a hydrate, a solvate, or a reduced form thereof.

In some embodiments, the second therapeutic agent is a chemotherapeutic agent. Preferably, the chemotherapeutic agent is a tumor immunotherapeutic agent. More preferably, the chemotherapeutic agent is an anti-PD-1 antibody, an anti-PD-L1 antibody, or a fragment thereof. In some embodiments, the second therapeutic agent is administered prior to, concurrently with, or subsequent to administration of the phenothiazine compound of formula I, or a hydrate, a solvate, or a reduced form thereof.

When the second therapeutic agent is not administered concurrently with the phenothiazine compound of the present invention, or a hydrate or solvate thereof, they are administered at an interval of about 0.1 hour to about 72 hours, for example, at an interval of about 0.1, 0.2, 0.3, 0.4, 0.5, 0.6, 0.7, 0.8, 0.9, 1, 2, 3, 4, 5, 6, 7, 8, 9, 10, 11, 12, 18, 24, 30, 36, or 72 h.

When the second therapeutic agent is administered concurrently with a phenothiazine compound of the present invention, a hydrate or solvate thereof, in some embodiments, the phenothiazine compound of the present invention, a hydrate, solvate or reduced form thereof and the second therapeutic agent are provided as separate pharmaceutical compositions. In some embodiments, the separate pharmaceutical compositions are provided in the same kit. In other embodiments, when the second therapeutic agent is administered concurrently with a phenothiazine compound of the present invention, a hydrate, solvate or reduced form thereof, the phenothiazine compound, a hydrate, solvate or reduced form thereof and the second therapeutic agent are provided in a single pharmaceutical composition.

Pharmaceutical Compositions and Kits

An aspect of the present invention provides a pharmaceutical composition for treating cancer, comprising a phenothiazine compound of formula I, or a hydrate, a solvate or a reduced form thereof,

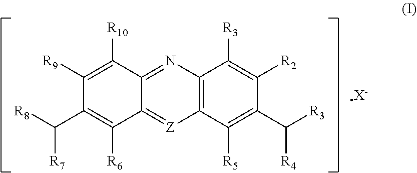

wherein,

Z is selected from a group consisting of S⁺, O⁺, C and N;

Y is N or N⁺; when Z is S⁺ or O⁺, Y is N; and when Z is C or N, Y is N⁺;

X⁻ is one or more anions that form a salt with Z⁺ or N⁺ to achieve electric neutrality; and

R₁, R₂, R₃, R₄, R₅, R₆, R₇, R₈, R₉ and R₁₀ are independently selected from a group consisting of hydrogen, substituted or unsubstituted alkyl, substituted or unsubstituted cycloalkyl, substituted or unsubstituted heterocyclic alkyl, substituted or unsubstituted aryl, substituted or unsubstituted heteroaryl, substituted or unsubstituted alkoxyl, substituted or unsubstituted arylalkoxyl, thioalkoxyl, amido, nitro, amino, and halogen.

In some embodiments, X is an inorganic anion or an organic anion. The inorganic anion is preferably Cl⁻, Br⁻ or I⁻. The organic anion is preferably methanesulfonic ion, ethanesulfonic ion, p-toluenesulfonic ion, benzenesulfonic ion, ethanedisulfonic ion, propanedisulfonic ion, or naphthalene disulfonic ion.

In some embodiments, the phenothiazine compound is 3,7-bis(dimethylamino)-phenothiazine-5-ium salt (MTX) having formula (II), or a hydrate, a solvate, or a reduced form thereof,

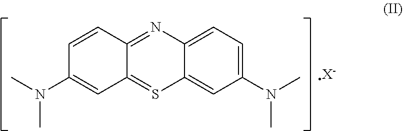

wherein X is one or more anion as defined above, to achieve electric neutrality.

In some embodiments, the reduced form of the phenothiazine compound is N3,N3,N7,N7-tetramethyl-10H-phenothiazine-3,7-diammonium salt (LMTX) having formula (III), or a hydrate or a solvate thereof,

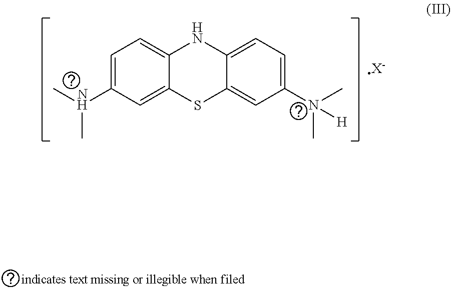

wherein X is one or more anion as defined above, to achieve electric neutrality.

In some embodiments, the phenothiazine compound is 3,7-bis(dimethylamino)-phenothiazine-5-ium salt (MTX), preferably 3,7-bis(dimethylamino)-phenothiazine-5-ium chloride (MTC).

In some embodiments, the phenothiazine compound is N3,N3,N7,N7-tetramethyl-10H-phenothiazine-3,7-diammonium dimesylate.

Another aspect of the invention provides a pharmaceutical composition for treating cancer, comprising a phenothiazine compound of formula I, or a hydrate, a solvate or a reduced form thereof; and an immune cell for adoptive cell therapy, wherein the formula I and substituents thereof are defined as above.

In some embodiments, the immune cell is selected from a group consisting of a tumor infiltrating lymphocyte (TIL), a chimeric antigen receptor T cell (CAR-T), a chimeric antigen receptor NK cell (CAR-NK) and a T cell receptor (TCR) chimeric T cell (TCR-T).

In some embodiments, the immune cell is a T cell receptor (TCR) chimeric T cell (TCR-T).

In some embodiments, the TCR binds peptide SIINFEKL.

Another aspect of the invention provides a pharmaceutical composition for treating cancer, comprising a phenothiazine compound of formula I, or a hydrate, a solvate or a reduced form thereof; and an immunotherapeutic agent for tumor chemotherapy, wherein the formula I and substituents thereof are defined as above.

In some embodiments, the immunotherapeutic agent for tumor chemotherapy is anti-PD-1 antibody, anti-PD-L1 antibody, or a functional fragment thereof.

Accordingly, the pharmaceutical composition of the present invention may comprise a single active ingredient (e.g., any of the compounds of formula I) present in the composition in admixture with a pharmaceutically acceptable carrier. The composition of the present invention may contain two active ingredients (for example, any one of the compounds of formula I and immune cells for adoptive therapy, or any one of the compounds of formula I and an anti-PD-1 antibody), which are contained in appropriate forms in the same pharmaceutical composition. When the pharmaceutical composition is administered, the subject is administered the two active ingredients simultaneously or sequentially. For example, the compound of formula I, the immune cells for adoptive cell therapy and the carrier are present in a mixture in the pharmaceutical composition at predetermined ratios. Alternatively, the compound of formula I and the carrier form a part of the pharmaceutical composition at a predetermined ratio, the immune cells for adoptive cell therapy and the carrier form another part of the pharmaceutical composition at a predetermined ratio, and the combination of the two parts constitutes the pharmaceutical composition for example in a core-shell structure. Other methods available in pharmacy or pharmaceutical engineering can also be used to combine the two active ingredients without affecting the effect of each active ingredient.

Another aspect of the present invention provides a kit comprising independently a first pharmaceutical composition and a second pharmaceutical composition, the first pharmaceutical composition comprising a therapeutically effective amount of immune cells suitable for adoptive cell therapy, the second pharmaceutical composition comprising a therapeutically effective amount of a phenothiazine compound of formula I or a hydrate or solvate thereof. Thus, in some embodiments of the kit, the first pharmaceutical composition can be presented in one separate dosage form and the second pharmaceutical composition can be presented in another separate dosage form, which dosage forms being the same or different. In some embodiments, the first pharmaceutical composition and the second pharmaceutical composition in the kit are each contained in separate containers.

Another aspect of the present invention provides a kit comprising independently a first pharmaceutical composition and a second pharmaceutical composition, the first pharmaceutical composition comprising a therapeutically effective amount of a phenothiazine compound of formula I or a hydrate or solvate thereof, the second pharmaceutical composition comprising a therapeutically effective amount of a second chemotherapeutic agent, e.g., an immunotherapeutic agent such as an anti-PD-1 antibody. Thus, in some embodiments of the kit, the first pharmaceutical composition can be presented in one separate dosage form and the second pharmaceutical composition can be presented in another separate dosage form, which dosage forms being the same or different. In some embodiments, the first pharmaceutical composition and the second pharmaceutical composition in the kit are each contained in separate containers.

A therapeutically or prophylactically effective amount of an active ingredient to be administered can be determined by standard procedures, taking into account factors such as the compound ICso, biological half-life, and age, size and weight of the subject, as well as conditions associated with the subject. The importance of these and other factors is well known to those of ordinary skill in the art. Generally, the dose will be between about 0.01 mg/kg and 50 mg/kg of the subject being treated, preferably between 0.1 mg/kg and 20 mg/kg.

Carriers or excipients can be used in the manufacture of pharmaceutical compositions. The carrier or excipient can be selected to facilitate administration of the compounds. Examples of carriers include calcium carbonate, calcium phosphate, various sugars (e.g., lactose, glucose or sucrose), starches, cellulose derivatives, gelatin, vegetable oils, polyethylene glycols and physiologically compatible solvents. Examples of physiologically compatible solvents include water for injection (WFI), saline solutions, and dextrose.

Suitable dosage forms depend in part on the route of administration, e.g., oral, transdermal, transmucosal, inhalation or by injection (parenteral). Such dosage forms should enable the active ingredient to reach target cells. The medicaments or pharmaceutical compositions of the present invention can be administered by different routes, including intravenous, intraperitoneal, subcutaneous, intramuscular, oral, transmucosal, rectal, transdermal, or inhalation. In some embodiments, oral administration is preferred. For oral administration, for example, the compounds can be formulated in conventional oral dosage forms, such as capsules, tablets, and liquid preparations, such as syrups, elixirs, and concentrated drops.

### EXAMPLES

The present invention will be described in further detail below with reference to specific examples.

The “MTC” in the following examples refers to a PD-1 signaling inhibitor, 3,7-bis(dimethylamino)-phenothiazine-5-ium chloride of formula

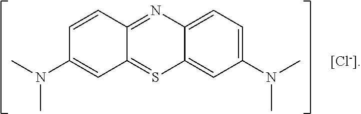

The “LMT” in the following examples refers to N3,N3,N7,N7-tetramethyl-10H-phenothiazine-3,7-diammonium dimesylate of formula,

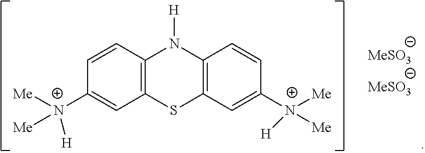

### Example 1. MTC Boosted Human T Cells Function

Methods

(1) Jurkat cells were used to construct a stable cell line expressing PD-1. The pCAGin-PD-1 plasmid was electroporated into Jurkat cells, which were then screened with 900 μg/mL G418 antibiotic and flow-sorted for single clones. Jurkat-PD-1 cells were then electroporated into a DNA fragment that controls the luciferase gene through the NFAT-binding site (referred to as NFAT-LUC). A single clone was then flow-sorted and named clone 5. Using this cell, the expression level of IL-2 can be measured by the activity of luciferase. Raji cells stably expressing PD-L1 were constructed, and the pCDNA-PD-L1 plasmid was electroporated into the Raji cells, followed by screening with 2 μg/mL puromycin, and then flow-sorted single clones and named clone 1.

(2) Raji cells stably expressing PD-L1 were used to stimulate PD-1 molecules on Jurkat-PD-1. Raji cells are human-derived B lymphocytes that express the B7 molecule, which provides the second signal required for T cell activation. Jurkat-PD-1-NFAT-LUC cells were stimulated with 2 μg/mL CD3 antibody/2 μg/mL CD28 antibody, then co-cultured with Raji-PD-L1 cells at a ratio of 1:1 for 6 hours. The phosphorylation at Y248 of PD-1 was detected by immunoblotting.

(3) A 96-well plate was coated with 10 μg/mL CD3 antibody/10 μg/mL CD28 antibody overnight at 4° C. and excess antibody was washed off with PBS. Raji cells or Raji-PD-L1 cells were co-cultured with Jurkat-PD-1-NFAT-LUC cells at a ratio of 1:1, or to a mixture of Raji-PD-L1 cells and Jurkat-PD-1-NFAT-LUC cells at a ratio of 1:1 was added 1 μM MTC or 10 μg/mL PD1 antibody for co-culture; after 6 hours of co-culture, the secretion of IL-2 in the supernatant was measured by ELISA.

(4) A 96-well plate was coated with 10 μg/mL CD3 antibody/10 μg/mL CD28 antibody overnight at 4° C., and excess antibody then washed off with PBS. Raji cells or Raji-PD-L1 cells were co-cultured with Jurkat-PD-1-NFAT-LUC cells at a ratio of 1:1, or to a mixture of Raji-PD-L1 cells and Jurkat-PD-1-NFAT-LUC cells at a ratio of 1:1 was added 4.86 μM MTC or LMT. Luciferase substrate was added after 6h co-culture and the luciferase activity was measured by a microplate reader.

Results and Analysis

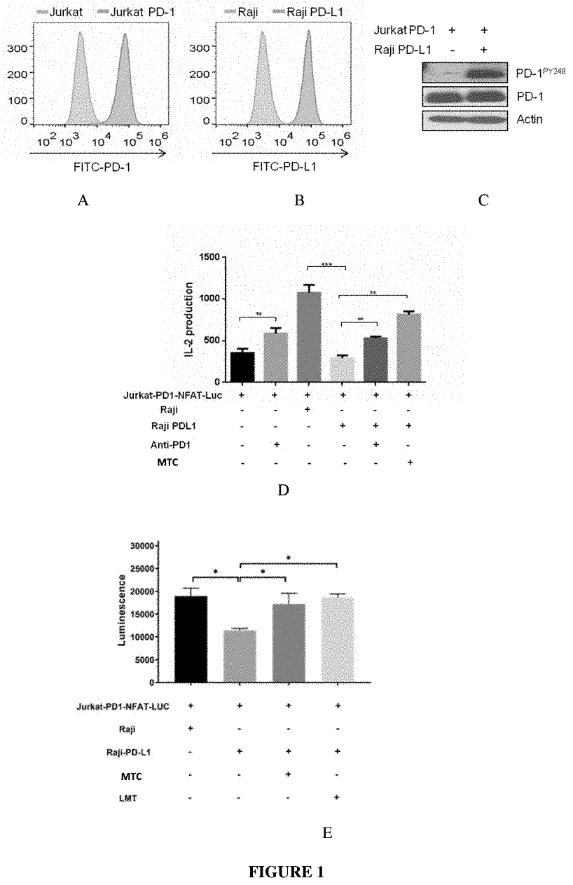

(1) Clone 5 Jurkat cell correctly expressed PD-1 molecule, as determined by FACS (FIG. 1A). Clone 1 Raji cell correctly expressed PD-L1, as determined by FACS (FIG. 1B).

(2) After Raji-PD-L1 cells were co-cultured with Jurkat-PD-1 cells for 6 hours, PD-1 was phosphorylated at Y248 as detected by immunoblotting (FIG. 1C). It showed that in the co-culture of Jurkat cells expressing PD-1 and Raji cells stably expressing PD-L1, PD-1 bound to PD-L1.

(3) IL-2 secretion by Jurkat-PD-1-NFAT-LUC was significantly stimulated by the addition of the anti-PD-1 antibody (Nivolumab, Bristol-Myers Squibb) or Raji cells. Compared with the Jurkat-PD-1-NFAT-LUC group without any stimulation, the secretion of IL-2 in the group added with Raji PDL1 decreased, but sharply enhanced when added with MTC or the PD-1 antibody (FIG. 1D). MTC significantly improved the function of Jurkat cells inhibited by PD-1 compared with the PD-1 antibody group.

(4) Jurkat-PD-1-NFAT-LUC had a limited increase in luciferase activity under the stimulation of CD3 antibody/CD28 antibody/Raji cells, while the activity of luciferase in cells added with Raji-PD-L1 decreased. When 4.86 μM MTC or LMT was added with Raji-PD-L1 cells, the activity of luciferase increased dramatically (FIG. 1E), indicating that MTC or LMT could improve the function of Jurkat cells inhibited by PD-1.

### Example 2. MTC Promoted OT-1 Cell Division and Secretion of Effector Molecules

The TCR expressed by CD8+ T cells of OT-1 transgenic mice recognizes the SIINFEKL-H-2 Kb complex of chicken ovalbumin. Therefore, when spleen cells of OT-1 mice are cultured in vitro, under the stimulation of SIINFEKL peptide, CD8+ T cells in the spleen can be activated and vigorously expanded, which are CTL cells.

Methods

(1) Preparation of CTL cells: The spleens of OT1 mice were obtained, ground and added with red blood cell lysate, to prepare single-cell suspension. The culture medium was 1640+10% FBS+50 μM βME+10 nM IL-2. Cell density was adjusted to about 2-4 million/mL. The cells were added with 10 nM OVA257-264, and cultured in a cell incubator at 37° C., 5% CO₂. From the day of CTL preparation, cells were stained with FITC-labeled PD-1 antibody every day, and the changes of PD-1 expression on the cell surfaces were detected by flow cytometry.

(2) The CTL cells were labeled with 5 nM CFSE, stimulated by 10 nM SIINFEKL and 10 nM IL-2, and added with 10 μg/mL mouse PD-L1 protein. The experimental group was also added with 100 nM MTC and stimulated for 24, 48, and 72 hours. The effects of PD-L1 protein and MTC on CTL cell division were observed by flow cytometry.

(3) CTL cells and target cells EG7 (EL4 lymphoma cells expressing chicken ovalbumin) were mixed and cultured at a concentration of 1:1. The cell mixture was treated with 1 μM protein transport inhibitor GolgiPlug™ for 6 hours, and then fixed by 4% paraformaldehyde. The cells were then treated with 0.1% saponin, and stained with antibodies against each effector molecule. The effect of MTC on the secretion of CTL effector molecules was detected by flow cytometry.

Results and Analysis

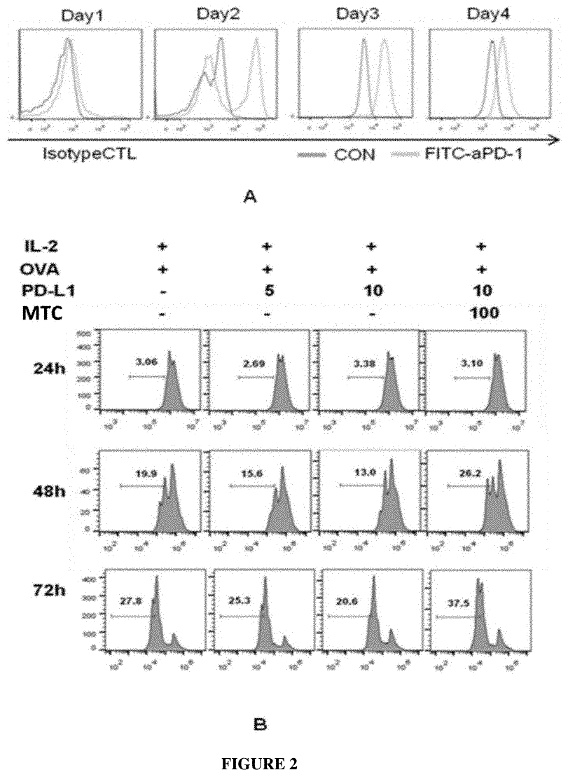

(1) The expression of PD-1 on the cell surface gradually increased over time during the entire induction of CTL cells (FIG. 2A).

(2) When CTLs were stimulated with SIINFEKL and IL-2, and PD-L1 protein was added, PD-L1 protein could inhibit the division of CTL cells, while the addition of 100 nM MTC reversed the inhibition (FIG. 2B).

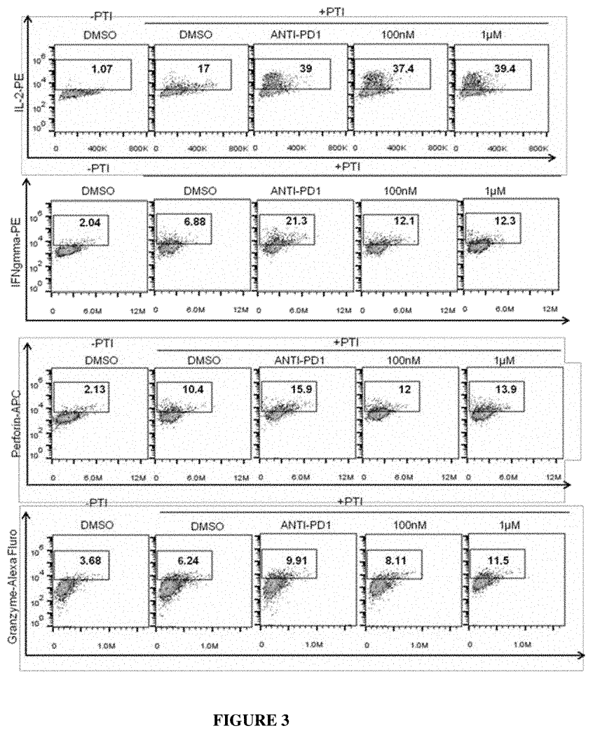

(3) EG7 is an EL4 lymphoma cell expressing chicken ovalbumin, so CTL cells can act its function of recognition and killing. When activated OT-1 mouse splenocytes and EG7 cells were mixed at a ratio of 1:1, CTL cells began to secrete effector molecules, such as IL-2, IFNγ, perforin, and granzyme B. EG7 cells overexpressing PD-L1 on the surface (EG7-PD-L1 stable cell line) strongly inhibited CTL secretion of IL-2, IFNγ, perforin, and Granzyme B by addition of protein transport inhibitor GolgiPlug™ (PTI), as detected by FACS. When treated with MTC, the secretion of IL-2, IFNγ, perforin, and Granzyme B in CTL cells increased sharply (FIG. 3), indicating that MTC can reverse the inhibition of PD-1 and promote the secretion of IL-2, IFNγ, perforin, and Granzyme B by CTL cells.

### Example 3. MTC Restored OT-1 T Cell Killing of Target Cells

Methods

(1) The preparation of CTL cells was described in Example 2.

(2) EG-7-PD-L1 cells were labeled with 5 nM CFSE. CTL cells and EG-7-PD-L1 were mixed at a ratio of 5:1, treated with 1 μM, 5 μM, and 10 μM MTC, respectively, for 4 hours, and then stained with 10 μg/mL PI. The apoptosis of target cells EG-7-PD-L1 was detected by flow cytometry, and then the killing ability of CTL cells to target cells was assessed.

(3) Melanoma cells B16-OVA (B16 cells expressing OVA gene) were treated with 10 μg/mL IFN-γ for 24 hours, and then mixed with CTL cells at a ratio of 1:5 and co-cultured for 4 hours. Cells were treated with 1 μM MTC. B16-OVA cells treated with DMSO or MTC only (without IFN-γ treatment) were used as control. The apoptosis of B16-OVA was observed by microscope, and then the killing ability of CTL cells to target cells was assessed.

(4) The B16-OVA group received no treatment, and the medium was replaced with water in the Water group. The other groups were treated with 10 μg/mL IFNγ. After 24 hours, except for the B16-OVA group, each group was co-cultured with CTL cells at a ratio of 1:5 for 4 hours, and treated with MTC (1 M), LMT (1 μM) or anti-PD1 (PD-1 antibody, 10 μg/mL, Nivolumab, BMS). The death of B16-OVA in each group was observed by microscope, and then the killing ability of CTL cells to target cells was assessed. Melanoma cell B16-WT group without genetical engineering did not receive any treatment. The IFN-γ+MTC group and the IFN-γ+LMT group were treated with 10 μg/mL IFN-γ for 24 hours. The cells were then mixed with CTL cells at a ratio of 1:5, co-cultured for 4 hours and treated with MTC (1 μM) or LMT (1 μM). The MTC and LMT groups were treated with MTC (10 μM) or LMT (10 μM) only. The killing ability against target cells was observed.

Results and Analysis

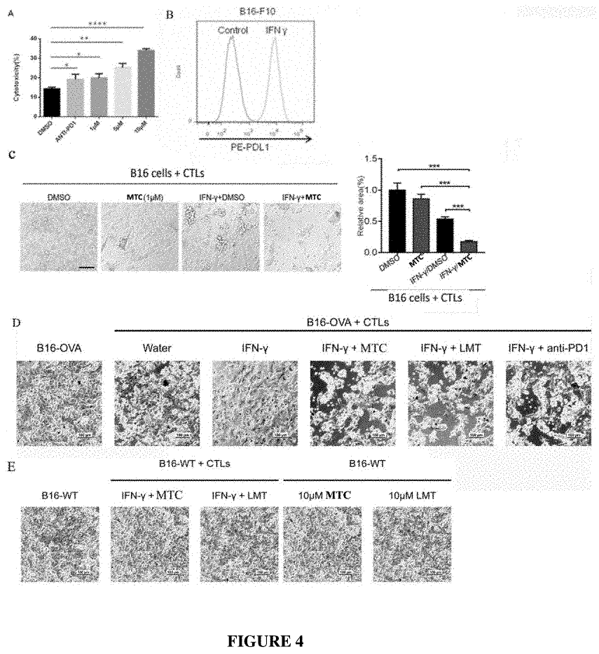

(1) Activated OT-1 cells (CTL cells) express PD-1 on the surface, so they killed EG-7-PD-L1 cells (OVA gene-modified mouse T lymphoma cells EG7 overexpressing mouse PD-L1) with limited capacity (FIG. 4A). In this system, MTC significantly restored the killing ability of CTL cells against EG-7-PD-L1 (FIG. 4A).

(2) As shown in FIG. 4E, MTC and LMT had no killing effect on B16-WT cells. Since OT-1 cells cannot recognize B16-WT cells, the combination of MTC/LMT and CTL cells had no killing effect on B16-WT cells. However, B16-OVA cells had low expression of MHC-I molecules, and the SIINFEKL peptides were not usually presented, so OT-1 cells cannot recognize and kill B16-OVA cells (FIGS. 4C and 4D). When B16-OVA cells were treated with IFNγ, the SIINFEKL peptide was presented, and CTL cells showed killing effect (FIGS. 4C and 4D). However, after IFNγ treatment, PD-L1 expression of B16-OVA was induced (FIG. 4B). Because the killing effect of CTL on B16-OVA was attenuated by the interaction between PD-1 and PD-L1, the killing effect was not significant when CTL was used alone. By adding MTC or LMT at the same time, the killing effects of CTL cells to B16-OVA cells under the above conditions was significantly enhanced (FIGS. 4C and 4D). In addition, as shown in FIG. 4D, the combined killing effect of MTC with CTL cells was comparable to that of LMT with CTL cells, and both was significantly stronger than that of the anti-PD-1 group. Therefore, the combination of MTC or LMT with CTL cells can synergistically enhance the killing effect of B16-OVA.

### Example 4. MTC Helps Cytotoxic T Cells Clear Tumors Formed by Target Cells

The results of Examples 1 to 3 confirmed that MTC can strongly restore cytotoxic T cell killing of PD-L1-overexpressing target cells. This example further evaluated the in vivo efficacy of MTC in treatment of tumors.

Methods

(1) Lymphoma cells overexpressing chicken ovalbumin OVA and mouse PD-L1, namely EL4-OVA-mPDL1 cells, were resuspended in BD Matrigel, in which, for one side of one mouse, 2×10⁶ cells were resuspended in 100 μl BD Matrigel.

(2) The mixture of cells and Matrigel was inoculated into the left and right flanks of Rag1−/− mice subcutaneously, each at a dosage of 100 μl.

(3) When the tumor was fixed about 5 days after inoculation, activated OT-1 T cells (i.e., 2×10⁶ CTL cells/mouse) were injected through the tail vein, and the mice were divided into 3 groups, namely the CTL cell group, the CTL cell +10 mg/kg/2 days PD-1 antibody group, and the CTL cell +40 mg/kg/day MTC group.

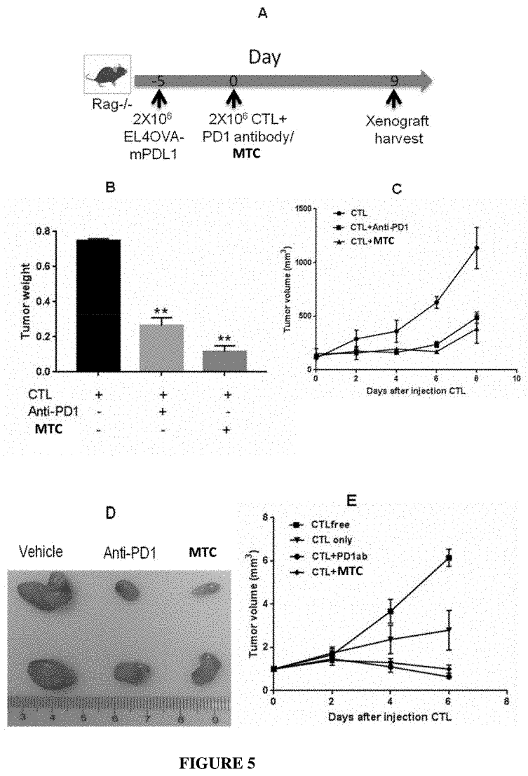

(4) The length and width of the transplanted tumor of each group of mice were measured from the day of tail vein injection of CTL cells, and every other day, and the tumor volume was calculated as length×width²/2 (FIG. 5A).

Results and Analysis

(1) Compared with the control group in which only CTL cells were injected through the tail vein, the tumor growth of the mice in the group additionally intraperitoneally injected with PD-1 antibody was inhibited. The rate at which tumor volume and tumor weight increased was both inhibited. The results showed that tumor growth in the PD-1 antibody group was inhibited.

(2) The intragastric administration of MTC to mice bearing xenograft also strongly inhibited tumor growth (FIGS. 5B, C, and D). After the treatment, the CTL cell-only group showed that the tumors of the mice grew rapidly, either indicated by the tumor weight or tumor volume, suggesting CTL alone could not effectively inhibit the tumor growth. However, both the CTL+PD-1 antibody treatment group and the CTL+MTC treatment group significantly inhibited tumor growth, and the CTL+MTC treatment group had better therapeutic effect than the CTL+PD-1 antibody treatment group.

Similar results were obtained in C57BL/6J mice with EG7-mPDL1 cells in a similar method to this example (FIG. 5E). The tumors in mice not given CTL (CTL free) grew rapidly. While CTL-only group inhibited tumor growth compared with CTL free group, the inhibitory effect was weaker than that of CTL+PD-1 antibody or CTL+MTC group. The results indicated that MTC can effectively help cytotoxic T cells clear subcutaneous tumors formed by target cells.

### Example 5. MTC can Effectively Treat In Situ Tumors

The advantages of xenograft models are that the antigenic epitopes recognized by T cells are very clear, and the system is pure for elucidation. The disadvantage is that this model cannot simulate the complex carcinogenesis, tumorigenesis, and interactions of tumor and stromal cells of the primary cancer. Therefore, murine xenograft models are less predictive of drug effects in human patients. Therefore, in this example, the effect of the MTC of the present invention in treating tumors was further investigated by using a transgenic lung cancer mouse model. By feeding EGFR-L858R transgenic mice with a diet containing tetracycline (DOX), the mouse lung epithelial cells can be induced to express human EGFR-L858R mutant which is commonly found in human lung cancer. One to two months later, lung cancer in mice could be detected and recorded by computed tomography (CT).

Methods

(1) The EGFR-L858R mutant mice were fed with DOX-containing diet to induce an orthotopic tumor model in about 40 days.

(2) The size and severity of the tumors was recorded by computed tomography (CT).

(3) The tumor-bearing mice were randomly divided into groups and given placebo (HKI solution) and MTC (40 mg/kg/day in HKI solution). After 2 weeks of treatment, tumor sizes were recorded by CT again.

(4) In order to prove that MTC removes tumors via CD8+ T cells, the following experiments were also carried out in this example. Mice were intraperitoneally injected with CD8 antibody (200 mg/mouse/3 days) one week in advance to deplete CD8+ cells in the tumor-bearing mice. The mice were then co-treated with MTC and CD8 antibody for 2 weeks, and the tumor size was recorded by CT.

Results and Analysis

The principle of this experiment lies in that when these mice are fed a diet containing DOX, rtTA in lung epithelial cells undergoes a conformational change after binding to DOX, and binds to the TetO sequence to initiate the expression of TetO-controlled EGFR mutants. These mice developed lung adenocarcinomas in situ after 40 days of feeding of tetracycline. These lung adenocarcinomas truly simulate the entire clinical process in which lung epithelial cells undergo carcinogenesis and development under the action of EGFR mutants, and the body finally dies from cancer.

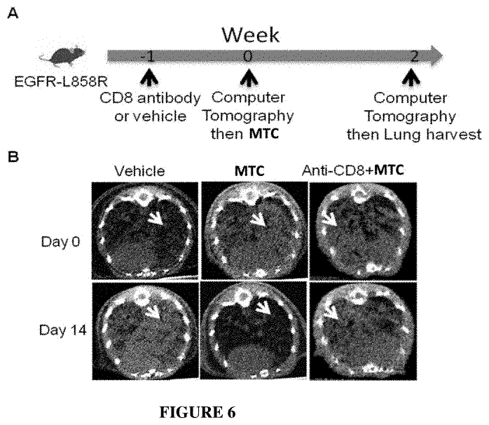

The experimental results of this example showed that the tumors in the placebo-treated group grew rapidly, while the imaging of the tumors in the mice treated by MTC for 14 days showed that the tumors were basically cleared (FIG. 6B). However, in the case of CD8+ T cell depletion, tumor growth was not inhibited in mice treated with MTC (FIG. 6B). Thus, MTC can effectively treat EGFR-L858R mutant orthotopic tumors by activating CTL in vivo.

### Example 6. MTC Blocked PD-1 Recruitment of SHP2

This example preliminarily explored the mechanism by which MTC inhibits PD-1 signaling, and assessed the ability of MTC to affect PD-1 recruitment of SHP2. The binding of PD-1 to ligands can recruit SHP2 protein to the surrounding T cell receptors, thereby inhibiting the activation of T cell receptor proximal kinases, reducing Lck-mediated phosphorylation of ZAP-70 protein and the initiation of downstream signaling pathways. Luciferase can be split into two fragments, N-terminal and C-terminal. When the two fragments are fused with other proteins, the activity of luciferase can be detected if the N-terminal and C-terminal luciferase fragments are close due to the interaction of their fusion proteins. Therefore, the strength of the interaction of the fused proteins can indirectly detected by measuring the activity of luciferase.

Methods

(1) Fusion genes PD-1-Cluc and Nluc-SHP2 were transfected into 293T cells with liposomes to construct a stable cell line. The cells were added with different concentrations of MTC and cultured for 6 hours to observe the effect of MTC on the reading of luciferase.

(2) Fusion genes PD-1-GFP and SHP2-mCherry were transfected into 293T cells with liposomes to construct a stable cell line. The cells were added with MTC at different concentrations, cultured for 6 hours, and then treated with 1 μM PVD (a dephosphorylase inhibitor) for 5 minutes. Cells were fixed with 4% paraformaldehyde and the effect of MTC on the localization of GFP and mCherry, namely PD-1 and SHP2, was observed under Confocal.

(3) The PD-1-Flag plasmid and SHP2 plasmid were co-transfected in 293T cells, and the cells were then treated with MTC at different concentrations. The ability of PD-1 to recruit SHP2 was detected by Co-IP.

Results and Analysis

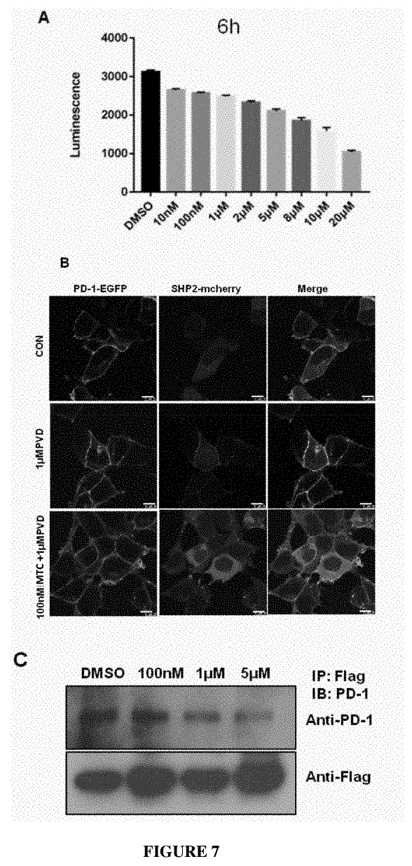

(1) Different concentrations of MTC were added to the 293T strains stably transfected with PD-1-Cluc and Nluc-SHP2. MTC could reduce the readings of luciferase, indicating MTC prevented PD-1 from recruiting SHP2 (FIG. 7A).

(2) 293T cells stably transfected with PD-1-GFP and SHP2-mCherry were used for confirming the previous results. GFP co-localized well with mCherry under the treatment of the inhibitor PVD when observed by Confocal, indicating that phosphorylated PD-1 recruited SHP2 (FIG. 7B).

(3) MTC addition in the experiment (2) resulted in the dissociation of mCherry from the membrane and showed its cytoplasmic distribution (FIG. 6B).

(4) Co-IP results showed that MTC prevented the binding of PD-1 to SHP2, unexpectedly blocked the downstream signaling of PD-1, thereby enhancing the function of immune cells, which in turn enhanced tumor cell killing effect when combined with adoptive cell therapy or other tumor immunotherapies (FIG. 7C).

The technical features of the above-described embodiments can be combined arbitrarily. For the sake of brevity, not all possible combinations of the technical features in the above-described embodiments are described. However, as long as there is no contradiction between the combinations of these technical features, these combinations should be regarded as within the scope of the present invention.

The examples described above are illustrative and should not be construed as a limitation on the scope of the present invention. It should be pointed out that for those skilled in the art, modifications and improvements can be made without departing from the concept of the present invention, which all belongs to the scope of the present invention. The scope of the present invention should be defined by the appended claims.

1. A pharmaceutical composition, comprising
(a)
a phenothiazine compound having formula (I), or a hydrate, a solvate or a reduced form thereof, 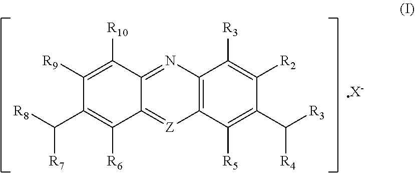
wherein,
Z is selected from a group consisting of S⁺, O⁺, C and N;
Y is N or N⁺; when Z is S⁺ or O⁺, Y is N; and when Z is C or N, Y is N⁺;
X⁻ is one or more anions that form a salt with Z⁺ or N⁺ to achieve electric neutrality; and
R₁, R₂, R₃, R₄, R₅, R₆, R₇, R₈, R₉ and R₁₀ are independently selected from a group consisting of hydrogen, substituted or unsubstituted alkyl, substituted or unsubstituted cycloalkyl, substituted or unsubstituted heterocyclic alkyl, substituted or unsubstituted aryl, substituted or unsubstituted heteroaryl, substituted or unsubstituted alkoxyl, substituted or unsubstituted arylalkoxyl, thioalkoxyl, amido, nitro, amino, and halogen, and
(b) a second therapeutic agent for cancer treatment.

2. The pharmaceutical composition of claim 1, wherein X⁻ selected from a group consisting of Cl⁻, Br⁻, I⁻, methanesulfonic ion, ethanesulfonic ion, p-toluenesulfonic ion, benzenesulfonic ion, ethanedisulfonic ion, propanedisulfonic ion, and naphthalene disulfonic ion.

3. The pharmaceutical composition of claim 1, wherein R₁, R₂, R₃, R₄, R₅, R₆, R₇, R₈, R₉ and R₁₀ are independently selected from a group consisting of hydrogen, substituted or unsubstituted C1-C6 alkyl, substituted or unsubstituted C3-C8 cycloalkyl, substituted or unsubstituted 3- to 8-member heterocyclic alkyl, substituted or unsubstituted C₅-C₁₀ aryl, substituted or unsubstituted 5- to 10-member heteroaryl, substituted or unsubstituted C₁-C₆ alkoxyl, substituted or unsubstituted arylalkoxyl, thioalkoxyl, amido, nitro, amino, and halogen.

4. The pharmaceutical composition of claim 1, wherein the phenothiazine compound is 3,7-bis(dimethylamino)-phenothiazine-5-ium salt (MTX) having formula (II), or a hydrate, a solvate, or a reduced form thereof, 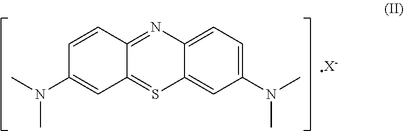
wherein X⁻ is one or more anion to achieve electric neutrality, or
N3,N3,N7,N7-tetramethyl-10H-phenothiazine-3,7-diammonium salt (LMTX) having formula (III), or a hydrate or a solvate thereof, 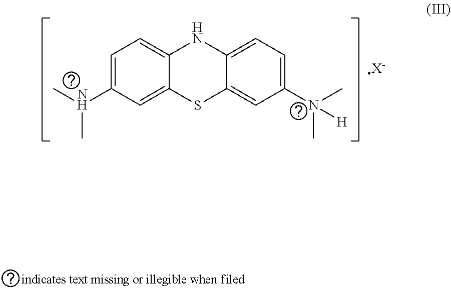
wherein X⁻ is one or more anion to achieve electric neutrality.

5. (canceled)

6. The pharmaceutical composition of claim 1, wherein the phenothiazine compound is 3,7-bis(dimethylamino)-phenothiazine-5-ium chloride, or N3,N3,N7,N7-tetramethyl-10H-phenothiazine-3,7-diammonium dimesylate.

7. (canceled)

8. The pharmaceutical composition of claim 33, wherein the immune cell is selected from a group consisting of a tumor infiltrating lymphocyte (TIL), a chimeric antigen receptor T cell (CAR-T cell), a chimeric antigen receptor NK cell (CAR-NK cell) and a T cell receptor (TCR) chimeric T cell (TCR-T cell).

9. The pharmaceutical composition of claim 8, wherein the immune cell is a T cell receptor (TCR) chimeric T cell (TCR-T cell).

10. The pharmaceutical composition of claim 9, wherein the TCR binds peptide SIINFEKL.

11. (canceled)

12. A method for treating cancer, for inhibiting PD-1 signaling, for boosting immune cell function, or for blocking PD-1 recruitment of SHP2 protein, comprising administering to a subject an effective amount of a phenothiazine compound of formula (I), or a hydrate, a solvate or a reduced form thereof, 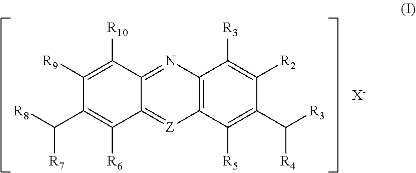
wherein,
Z is selected from a group consisting of S⁺, O⁺, C and N;
Y is N or N⁺; when Z is S⁺ or O⁺, Y is N; and when Z is C or N, Y is N⁺;
X⁻ is one or more anions that form a salt with Z⁺ or N⁺ to achieve electric neutrality; and
R₁, R₂, R₃, R₄, R₅, R₆, R₇, R₈, R₉ and R₁₀ are independently selected from a group consisting of hydrogen, substituted or unsubstituted alkyl, substituted or unsubstituted cycloalkyl, substituted or unsubstituted heterocyclic alkyl, substituted or unsubstituted aryl, substituted or unsubstituted heteroaryl, substituted or unsubstituted alkoxyl, substituted or unsubstituted arylalkoxyl, thioalkoxyl, amido, nitro, amino, and halogen.

13. The method of claim 12, wherein the phenothiazine compound is 3,7-bis(dimethylamino)-phenothiazine-5-ium salt (MTX) having formula (II), or a hydrate, a solvate, or a reduced form thereof, 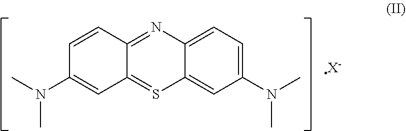
wherein X⁻ is one or more anion to achieve electric neutrality, or
N3,N3,N7,N7-tetramethyl-10H-phenothiazine-3,7-diammonium salt (LMTX) having formula (III), or a hydrate or a solvate thereof, 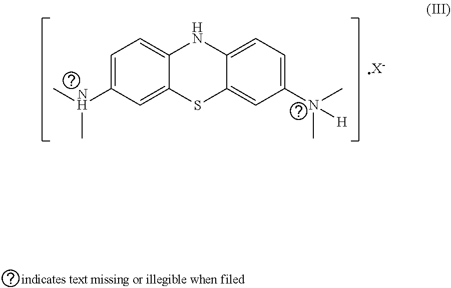
wherein X⁻ is one or more anion to achieve electric neutrality.

14. (canceled)

15. The method of claim 12, wherein the phenothiazine compound is 3,7-bis(dimethylamino)-phenothiazine-5-ium chloride, or N3,N3,N7,N7-tetramethyl-10H-phenothiazine-3,7-diammonium dimesylate.

16. (canceled)

17. The method of claim 12, wherein the cancer is selected from a group consisting of melanoma, thymic tumor, lung cancer, prostate cancer, breast cancer, ovarian cancer, colorectal cancer, pancreatic cancer, liver cancer, lymphoma, esophageal cancer, bladder cancer, urethral cancer, non-Hodgkin's lymphoma, kidney cancer and brain tumor.

18-20. (canceled)

21. The method of any of claim 12, wherein X⁻ selected from a group consisting of Cl⁻, Br⁻, I⁻, methanesulfonic ion, ethanesulfonic ion, p-toluenesulfonic ion, benzenesulfonic ion, ethanedisulfonic ion, propanedisulfonic ion, and naphthalene disulfonic ion.

22. The method of claim 12, wherein R₁, R₂, R₃, R₄, R₅, R₆, R₇, R₈, R₉ and R₁₀ are independently selected from a group consisting of hydrogen, substituted or unsubstituted C₁-C₆ alkyl, substituted or unsubstituted C₃-C₈ cycloalkyl, substituted or unsubstituted 3- to 8-member heterocyclic alkyl, substituted or unsubstituted C₅-C₁₀ aryl, substituted or unsubstituted 5- to 10-member heteroaryl, substituted or unsubstituted C₁-C₆alkoxyl, substituted or unsubstituted arylalkoxyl, thioalkoxyl, amido, nitro, amino, and halogen.

23-29. (canceled)

30. The pharmaceutical composition of claim 1, wherein the second therapeutic agent is a tumor immunotherapeutic agent.

31. The pharmaceutical composition of claim 30, wherein the tumor immunotherapeutic agent is an anti-PD-1 antibody, an anti-PD-L1 antibody, or a functional fragment thereof.

32. A kit for treatment of cancer, comprising
(a) a phenothiazine compound formulated into a first formulation, wherein the phenothiazine compound is a compound of formula (I), or a hydrate, a solvate or a reduced form thereof, 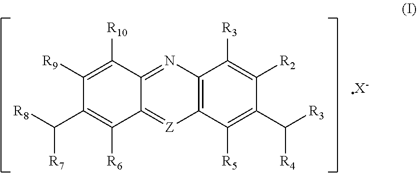
wherein,
Z is selected from a group consisting of S⁺, O⁺, C and N;
Y is N or N⁺; when Z is S⁺ or O⁺, Y is N; and when Z is C or N, Y is N⁺;
X⁻ is one or more anions that form a salt with Z⁺ or N⁺ to achieve electric neutrality; and
R₁, R₂, R₃, R₄, R₅, R₆, R₇, R₈, R₉ and R₁₀ are independently selected from a group consisting of hydrogen, substituted or unsubstituted alkyl, substituted or unsubstituted cycloalkyl, substituted or unsubstituted heterocyclic alkyl, substituted or unsubstituted aryl, substituted or unsubstituted heteroaryl, substituted or unsubstituted alkoxyl, substituted or unsubstituted arylalkoxyl, thioalkoxyl, amido, nitro, amino, and halogen; and
(b) a second therapeutic agent for cancer treatment, formulated into a second formulation.

33. The pharmaceutical composition of claim 1, wherein the second therapeutic agent is an immune cell for adoptive cell therapy.

34. The kit of claim 32, wherein the second therapeutic agent is an immune cell for adoptive cell therapy.

35. The kit of claim 32, wherein the second therapeutic agent is an anti-PD-1 antibody, an anti-PD-L1 antibody, or a functional fragment thereof.
